(19) United States
(12) Patent Application Publication
(10) Pub. No.: US 2024/0065412 A1
(43) Pub. Date: Feb. 29, 2024

(54) APPLICATOR AND METHOD FOR APPLYING A LIQUID TO SKIN OR HAIR

(71) Applicant: Kevin D. Young, Philadelphia, PA (US)

(72) Inventor: Kevin D. Young, Philadelphia, PA (US)

(73) Assignee: Kevin D. Young, Philadelphia, PA (US)

(21) Appl. No.: 18/237,254

(22) Filed: Aug. 23, 2023

Related U.S. Application Data
(60) Provisional application No. 63/400,123, filed on Aug. 23, 2022.

(51) Int. Cl. A45D 24/22; A45D 34/04; A46B 7/04; A46B 9/00; A46B 11/00; A46B 15/00; A46D 1/00

(52) U.S. Cl. CPC A45D 24/22; A45D 34/042; A46B 7/04; A46B 9/005; A46B 11/0006; A46B 11/0072; A46B 15/0055; A46D 1/0246; A45D 2200/054; A46B 2200/1033; A46B 2200/1046; A46B 2200/1093

(57) ABSTRACT

Generally, this invention is an applicator that provides for easy and efficient application of liquid substances to skin and/or hair; preferably, to a person's body, hair or skin, or to a pet's body, hair or skin. The applicator includes a handle portion having an engagement portion, a liquid medicine dispenser that removably engages with the handle portion's engagement portion, the dispenser comprises a hollow interior space with a liquid substance, preferably medicinal and at least one opening for dispensing the liquid substance through hollow “teeth” connected to the handle portion. The teeth selectively distribute the liquid substance to the person or pet's hair by brushing. In another embodiment, the applicator is made of a material that is easily disposable or breakable so that it is safe for use with children or in potentially violent atmospheres such as prisons.

### CROSS-REFERENCE TO RELATED APPLICATIONS

This application claims the benefit of U.S. Provisional Application No. 63/400,123, filed 23 Aug. 2022 and incorporated by reference in its entirety.

### FIELD OF THE INVENTION

The present invention relates generally, to the field of devices for easily and efficiently applying liquid sub-stances to skin and/or hair, specifically, to applicator systems for applying liquid products to a person's body, hair or skin, or to a pet's body, hair or skin. The present invention further relates to an applicator that includes a safety feature.

### BACKGROUND OF THE INVENTION

It is often necessary to apply medicinal substances or other liquid substance to a person's body, hair, whether it is the person's scalp or body hair. As used herein, the term “liquid substance” or “medicinal substance” or “medicinal liquid” or “medicated liquid” refers to any liquid, gel, shampoo, salve, suspension, emulsion, or other vehicle for delivering pharmaceutical agents, medicine, antibiotics, nutraceuticals, supplements, vitamins, or similar substances, to the hair and/or skin of a person, or the coat and/or skin of a pet or other animal. As used herein, the term “pharmaceutical agent” refers to any drug. Liquid substances can be used for treating various ailments, such as body lice, dermatitis, dandruff, infections, and many other diseases of the hair, body and skin. Liquid substances can also be used for general hair or skin care, or health and beauty needs, such as for treating the appearance of the hair or skin.

In some situations, such as with certain adults or children, it is difficult to entrust an applicator made of a hard material, such as a hard plastic or metal, to the individual. For example, an adult that has been incarcerated would not be entrusted with an applicator made of a hard material such as a hard plastic, which could be used as or fashioned into a weapon. Due to safety concerns, an alternate applicator would be preferable or more preferably an applicator having a more precise control of the application of use. Particularly in the field of pet care, it is often necessary to apply a liquid substance to a pet's coat or skin, for various reasons. Difficulties in applying a liquid substance to a pet's coat or skin include ensuring that the liquid substance spread uniformly over the pet's coat, or contact to the pet's skin. Often, liquid substances are supplied in bottles, tubes or jars, and are applied to the coat of a pet. Then, a brush or comb is used to spread the liquid substance throughout the pet's coat or skin. This is inefficient, and does not spread the liquid substance uniformly. Also, it may be necessary to use an applicator for applying a liquid sub-stance to a child's hair and/or body, such as in the case of an outbreak of lice.

Thus, there is the need for a device that will easily, efficiently, and generally uniformly spread liquid substances on and through a person's or pet's hair and/or skin.

There is further the need for an applicator that is formed with safety in mind.

### SUMMARY OF THE INVENTION

The present invention is an applicator comprising: a handle portion having an engagement portion; a dispenser comprising: a hollow interior space for holding a liquid substance, a liquid substance held at least partially within the hollow interior space, at least one opening for dispensing the liquid substance, and, an engagement portion; the engagement portion of the dispenser removably engagable with the engagement portion of the handle portion.

### BRIEF DESCRIPTION OF THE DRAWINGS

FIG. 1 is a cross sectional side view of an applicator according to the present invention.

FIG. 2 is a close up cross-sectional view of one dispenser tooth according to the present invention.

FIG. 3 is a cross sectional view of the applicator shown, with the dispenser cartridge disengaged from the handle portion.

FIG. 4 is a close up partial cross-sectional view of a tooth of the dispenser of the present invention.

FIG. 5 is a close up partial cross-sectional view of an alternate embodiment of a tooth of the dispenser of the present invention.

FIG. 6 is a cross sectional side view of another embodiment of an applicator according to the present invention.

FIG. 7 is a cross sectional side view of another embodiment of an applicator according to the present invention.

FIG. 8 is a cross sectional side view of another embodiment of an applicator according to the present invention.

FIG. 9 is a cross sectional side view of another embodiment of an applicator according to the present invention.

FIG. 10 is a bottom view of another embodiment of an applicator according to the present invention.

FIG. 11 is a perspective view of a disposable and breakable applicator according to another embodiment of the present invention.

FIG. 12 is a cross sectional view of the disposable and breakable applicator according to the present invention.

FIG. 13 is a cross sectional view of another embodiment of the disposable and breakable applicator according to the present invention.

FIG. 14 is a cross sectional view of the disposable and breakable applicator according to the present invention, including a cover.

FIG. 15 is a cross sectional view of the disposable and breakable applicator according to the present invention, including a plastic closure sheet.

FIG. 16 is a perspective view of a disposable and breakable applicator according to the present invention, showing the applicator fragmented after force has been applied to the applicator.

FIG. 17 is a cross sectional view of another embodiment of a disposable and breakable applicator according to the present invention.

FIG. 18 is a perspective view of another embodiment of a disposable and breakable applicator according to the present invention, showing the applicator including fragmentation lines along which the applicator can easily break.

FIG. 19 is a top view of the receiving brush head having an interlocking engagement portion with an ergonomic handle grip.

FIG. 20 is magnified view of the hollow brush teeth containing the saturated felt tip.

FIGS. 21A and 21B is a perspective view of securing cap having the felt packet with connected felt teeth in Panel A showing corresponding insert attachment portion in Panel B.

FIG. 22 is a still further perspective view of the solid rigid, disposable and removable attachment with the felt packet insert for receiving the solid handle.

### DESCRIPTION OF THE INVENTION

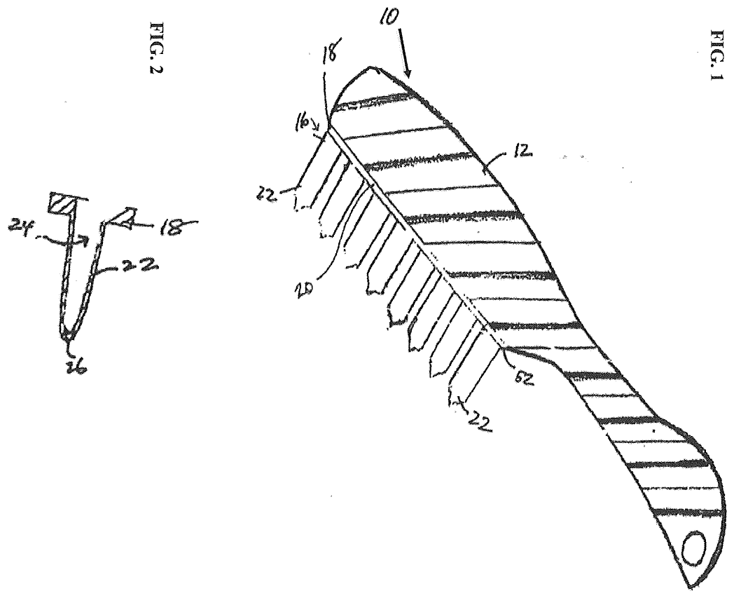

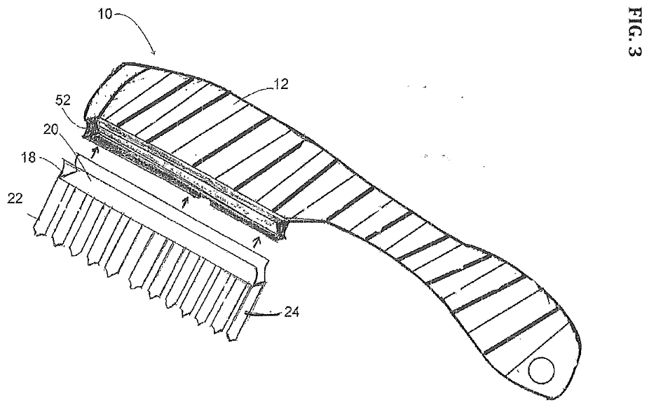

FIG. 1 and FIG. 2 show one embodiment of an applicator 10 according to the present invention. The applicator 10 includes a handle portion 12 having at least one opening 14 for receiving a dispenser 16. In a preferred embodiment of the present invention, the dispenser 16 is prefilled with, for example, a liquid substance for applying to a person's hair or skin, or a pet's coat or skin. In that preferred embodiment, the applicator is shaped as a hair brush or comb; however, other shapes may be used. The applicator may be formed with or without a handle portion, depending on preference.

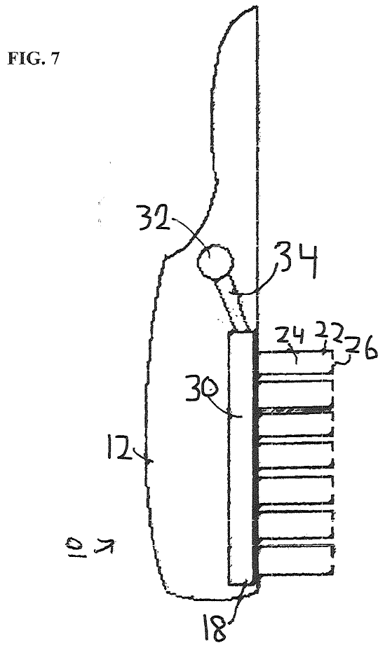

The dispenser 16 of the present invention may take several forms. In one embodiment, the dispenser 16 is formed as a cartridge 18. The cartridge 18 is a single unit formed having an interior portion 20 (See FIG. 7) which is at least partially hollow. A liquid substance including, for example, a drug in a liquid vehicle, is provided in the interior portion 20 as shown in FIG. 3. The drug may be any known drug, such as a pharmaceutical, a homeopathic drug, a vitamin, or nutraceutical drug, or any other substance for the treatment, mitigation, or cure of disease in man or other animals. The handle portion 12 is formed including an engagement portion 52 that is adapted to removably engage the cartridge 18. The cartridge 18 may be formed having ridges, and the engagement portion 52 may be formed having corresponding flanges for engaging the ridges, so that the cartridge 18 may slide into place and attach to the handle portion. Alternately, the handle and cartridge 18 may be formed of plastic, whereby the cartridge 18 may be formed to snap-in and engage the engagement portion 52. Other arrangements whereby the cartridge 18 may be selectively attached to and disengaged from the handle portion may be used without departing from the present invention.

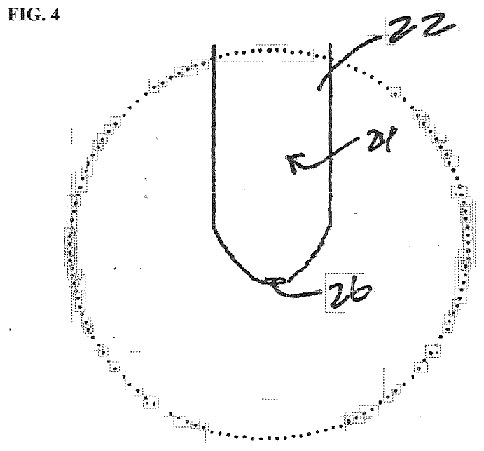

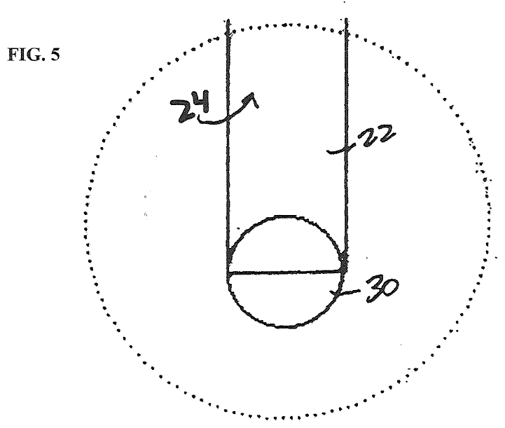

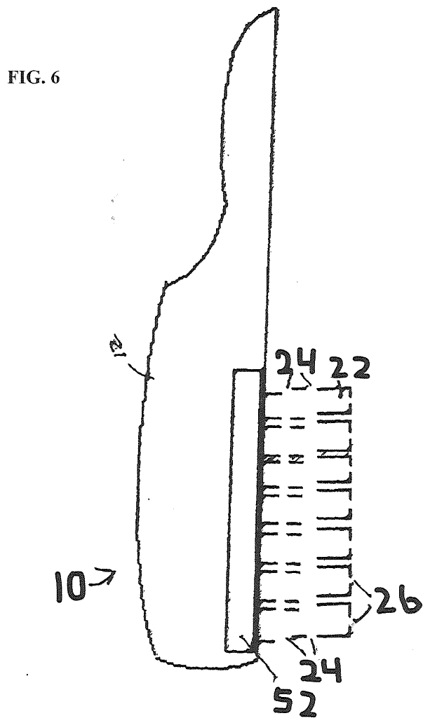

The cartridge 18 has a plurality of teeth 22 extending therefrom. At least one tooth 22 has a hollow interior portion or space 24 adapted to receive the medicinal liquid. As shown in FIG. 2, FIG. 4, and FIG. 5, an opening 26 is provided at the application end 26 of each tooth 22 for allowing liquid to pass from the interior portion 24 to the coat of skin of a pet or human. The teeth may have multiple openings 26 for allowing the dispensing of the liquid, as shown in FIG. 6.

The application end 26 of each tooth 22 may be formed in any manner whereby the liquid substance held within the interior portion 24 of the tooth 22 will be dispensed to and/or contact the coat or skin of a pet as needed when the applicator is in use. For example, the application end 26 may be formed having a permeable, semipermeable, absorptive, or adsorptive plug 30 for controlling the passage of liquid, as shown in FIG. 5. The plug 30 may be formed from a material adapted to absorb or adsorb the liquid substance, such as a cloth material, felt, gauze, micropores, cotton, coated plastic, or other similar materials. The material forming the plug 30 may be selected so that the liquid substance is dispensed at a controlled and uniform rate. Those familiar with the art of liquid substance applicators, particularly in the field of pharmaceutical applicators, will be familiar with such materials.

The plug 30 may be formed as a “rolling ball” arrangement, such as in a ballpoint ink pen, where the plug 30 rotates as the applicator 10 is passed over a person's hair or skin or pet's coat or skin. The plug 30 will roll or rotate during application, becoming coated with the liquid substance from a side facing the interior portion 24, and applying the liquid substance to a person's hair or skin, or a pet's coat or skin, when it contacts the person's hair or skin or, the pet's coat or skin. This arrangement provides for a comfortable and uniform application method.

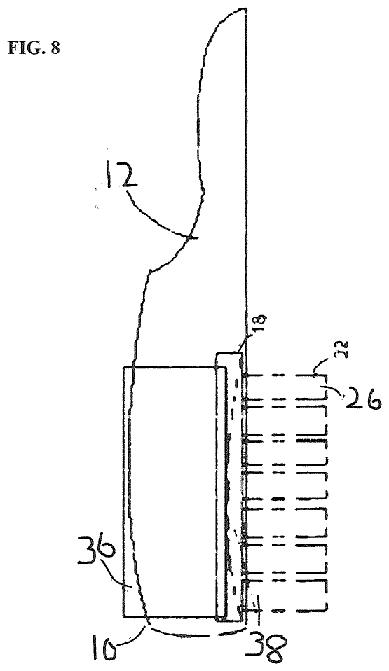

The handle portion 12 may be formed having a means for selectively dispensing the liquid substance from the interior portions 20, 24, and out of the openings in the teeth 22, as shown in FIG. 7 and FIG. 8. For example, an actuator such as a button 32 may be provided and may be formed as a reservoir 34 in communication with the interior portion 20. Depressing or squeezing the button 32 will contract the reservoir 34, and provide a pressurized or mechanical force to urge the liquid substance out of the interior portion 24 and through the teeth 22.

Alternately, the actuator may be a push bar 36, as shown in FIG. 8, may be provided extended from the back of the handle portion 12. The push bar 36 is moveable, so that it can press against, for example, a bladder or reservoir 38 formed in the interior portion of the cartridge 18. When a user presses on the push bar 36, force is exerted on the bladder or reservoir 38, forcing more liquid substance from the teeth 22. It is appreciated that other arrangements for selectively increasing the amount of liquid substance dispensed, or the force with which the liquid substance is dispensed, may be employed without departing from the current invention.

For ease of use and efficiency, the cartridge 18 is detachable from the handle portion 12, and can be easily replaced with another cartridge 18. Thus, the cartridge 18 may be sold separately from the handle portion 12, and may be prefilled with any liquid substance, or with a combination or plurality of liquid substances. A user may purchase the handle portion 12 adapted for use in connection with the present invention, and simply purchase replacement cartridges as needed. In addition, the cartridge 18 may be prefilled with different liquid substances, such as combinations or different medicinal liquids (for pets, antiflea, antitick, vitamins; for people, antibacterials, antifungus, antidandruff, etc.) in different teeth.

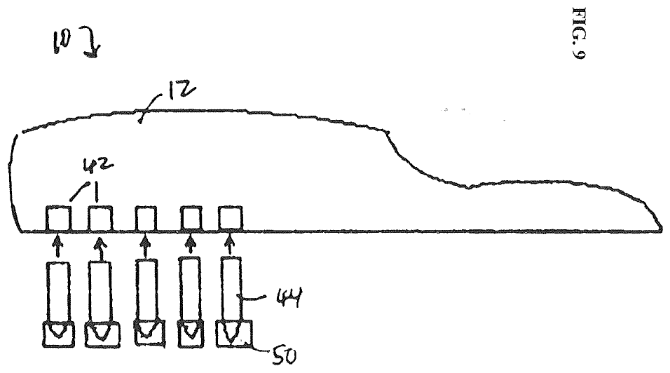

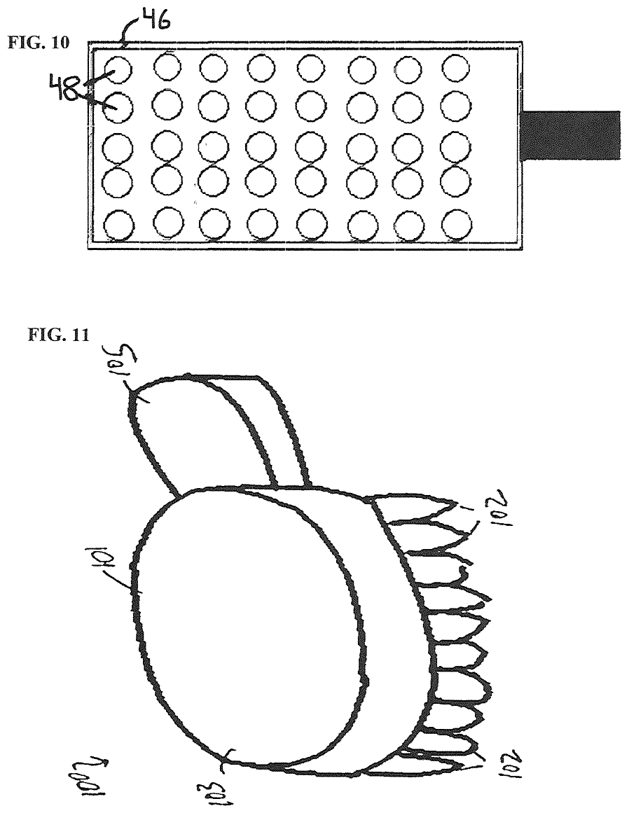

In an alternate embodiment shown in FIG. 9, rather than having an entire cartridge 18 as the dispenser 16, a handle 40 may have engagement sections formed as openings 42 for receiving individual ampules 44. The ampules 44 act as the teeth of a comb or brush. Ampules 44 are frictionally held within the openings 42, and may be held by a snapping arrangement or a tongue-in-groove arrangement. Shown in FIG. 10 is an alternate embodiment showing a brush handle 46 formed having openings 48 for receiving the ampules 44. The ampules 44 may be formed having caps or other stopper elements to maintain the liquid substance within the ampules 44 prior to use. The ampules 44 may be formed having an opening, a plurality of openings, a plug as described above, or other similar arrangements for dispensing the liquid substance held within the ampule 44 when the cap is removed. The ampules 44 may include a peal-away plastic cover portion so that the liquid substance will not leak prior to use.

According to this embodiment, it is possible for a user to selectively arrange different user selected liquid substances in the handle 40 for applying to a person's hair or skin or, a pet's coat or skin. The ampules 44 may be provided prefilled with different liquid substances. Thus, a combination of medications (ampules for psoriasis, ampules for dandruff, ampules with liquid vitamin E, etc.) may be applied at once, all based on a user's preference.

As is apparent, the present invention provides a use with many options. Different liquid substances can be selected for use with the handle portions, and can be arranged as needed by the user. The applicator of the present invention is easy to use, and effective in dispersing a liquid substance on a subject for use with a person's hair or skin or pet's coat or skin, if the subject is a person or pet. The dispenser provides for ease of use and easy clean-up. In addition, because the liquid substance is not stored in the handle, it can be stored safely and will not evaporate or otherwise spoil. The handle may be formed in different ergonomic shapes, or having extensions for applying liquid substance to different parts of a pet.

The shape may be optimized for a person's hair or skin or pet's coat or skin. Each optimization requires a protocol for use which can include a direction along the hair or skin. The direction can be a single direction or back-and-forth motion, depending upon the optimal dispersal conditions of the liquid.

In the area of pet care, the invention of the present device has applications in domesticated mammals, cattle, horses including any animal with fur, hair, wool or feathers thereof. The device is useful to uniformly spread pest control emulsions or liquids and other fluids to eradicate infections, such as but not limited to fleas, ticks, biting flies. The device will uniformly spread skin care medicated liquids, salve, etc to address ‘mange’ or other dermatological conditions for pets such as dogs, cats, rabbits, or other mammals, such as cattle, horses, etc. Further uses include uniformly spreading skin care liquid vitamins, supplements directly to animals skin or scalp under the hair or fur in order to restore PH balance to ordinarily dry, flaky skin and in order to control dander which is usually what makes some pet owners or others allergic to said animal. Such dander can be reduced down dramatically, if not controlled completely. The device is also useful to uniformly spread skin care liquid vitamins, supplements directly to animals skin or scalp under the hair or fur in order to restore PH balance to ordinarily dry, flaky skin in order to lessen shedding due to overly dry, unhealthy skin. Such shedding often precludes would be pet owners from owning, adopting pets.

A disposable and breakable applicator 100 is also disclosed herein, that is at least partially formed from a material that meets the following properties: the material should not be capable of being formed into a sharp or dangerous object; the material should be readily breakable, frangible and/or deformable when a sufficient force is applied to it, such as if an attempt is made to shape or sharpen it into a weapon or if the applicator is used in an attempt to hurt a person or animal; the material should be capable of being formed or molded into the shape of an applicator as described herein; the material should be strong and dense enough for packing, shipping and/or containing a liquid substance without breaking during normal shipping conditions and normal use. In a preferred embodiment, the entire applicator 100, or a majority of the body of the applicator 100, is formed from a suitable material meeting the previously listed criteria.

Suitable materials for forming at least a portion of the applicator may include, but are not limited to: foam, styrofoam, polystyrene foams, polymethacrylimide foam (Rohacell 51), polyvinylchloride foam (Conticell), polyurethane foam, Styrofoam IB, polypropylene foam (EPP), Nomex Honeycomb, rigid urethane foam, polyethylene foam, ionomer foam, acrylonitrile butadiene styrene, neoprene, polybutadiene rubbers, or equivalents or substitutes of any of these materials. Rubber or silicone may also be used. Combinations of any of the previously listed materials may also be used.

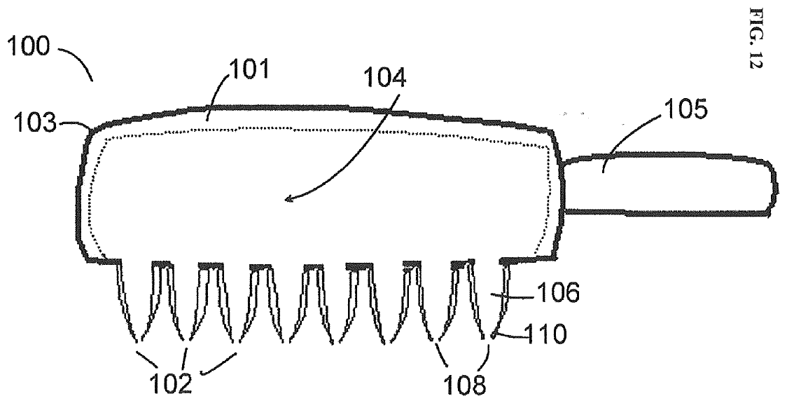

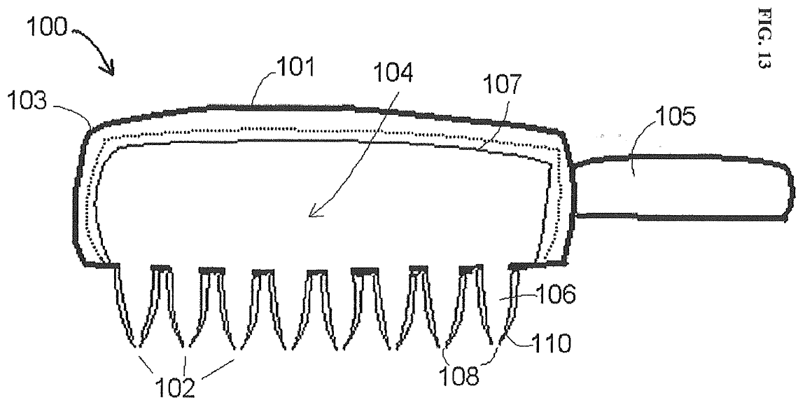

One embodiment of the disposable and breakable applicator 100, is formed from one or any combination of the listed suitable disposable and breakable materials, such as a Styrofoam. The applicator 100 may take the form of a brush 101 or comb, including a brush body 103 and handle 105, as shown in FIG. 11 and FIG. 12, and having a plurality of teeth 102. The brush 101 is formed having a cavity 104 for storing a liquid substance.

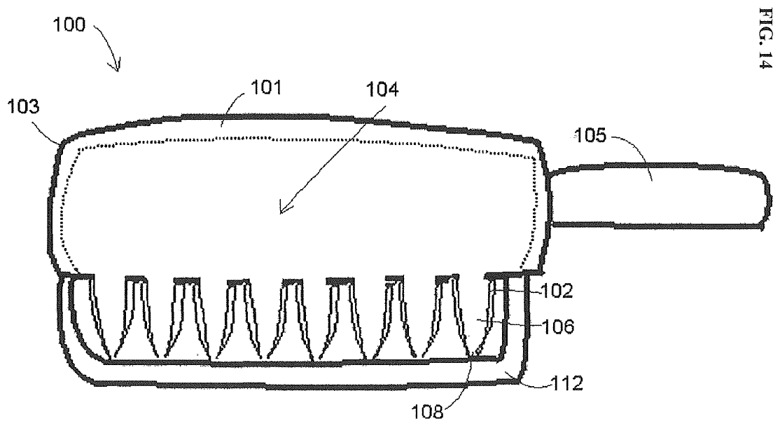

In one embodiment, shown in FIG. 13, the cavity 104 is lined, coated or otherwise treated with a material, such as a plastic 107 or other coating, so that the liquid substance does not eat away or otherwise damage the inner walls of the applicator 100 forming the cavity 104.

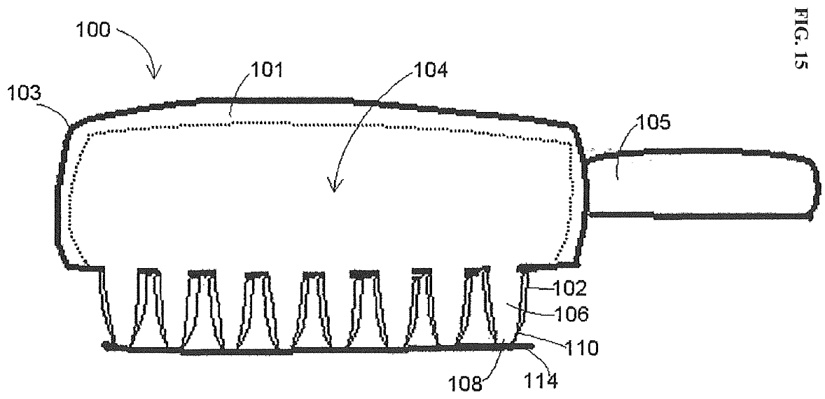

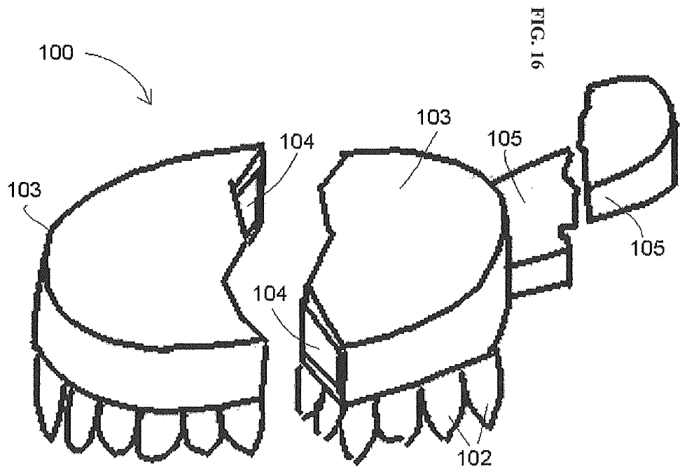

At least one tooth 102 is formed having a reservoir 106 that may be in communication with the cavity 104. A tooth 102 formed with such a reservoir 106 has an opening 108 at its end 110, so that a liquid substance in the cavity 104 may flow through the reservoir 106, and out of the opening 108 to treat, for example, a person's skin or hair, or a pet's skin or coat. A select number or all of the teeth 102 may be formed having such reservoirs 106 and openings 108. The inner walls of the teeth 102 forming the reservoirs 106 may be treated with or coated with a material, such as a plastic, so that the liquid substance does not eat away or otherwise damage the inner walls of the teeth 102. As shown in FIG. 14, a cover 112 may be provided covering and sealing the openings 108 of the applicator 100 prior to use. A plastic peal-away film or sheet may also be provided which is pealed off before use. The cover 112 may be formed of the same material as the brush 101, or any other safe material. The cover 112 may removably attach to the brush body 103 such as through a tongue and groove arrangement, by an adhesive, by hook and loop fasteners, snapping engagement, or any other acceptable attachment means. Alternately, as shown in FIG. 15, a plastic film or sheet 114 may be provided sealing the openings 108 of the applicator 100 prior to use. The sheet 114 may be attached adjacent the opening 108 such as by an adhesive. When ready for use, the sheet 114 is pealed away and disposed of, and any liquid substance stored in the cavity 104 may pass through the reservoirs 106 and out of the openings 108.

In use, the cover 112 or sheet 114 is removed from the applicator 100, and the applicator 100 is used to supply the liquid substance until the treatment is complete, or the liquid substance runs out. The present invention provides a discrete amount of liquid substance (such as an amount that is non-lethal or non-toxic even if ingested), and a disposable applicator 100.

If, for example, an inmate at a prison attempts to fashion the applicator 100 into a make-shift weapon, because of the properties of the material(s) from which the applicator 100 is made, the applicator 100 will simply break or crumble into pieces, as shown in FIG. 16.

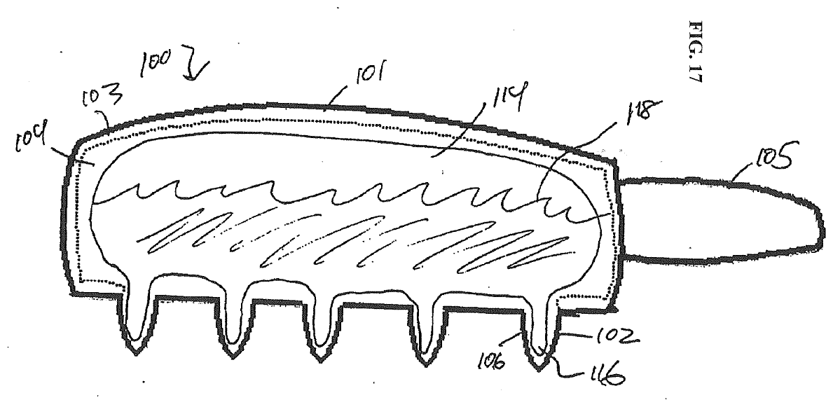

In an alternate embodiment, as shown in FIG. 17, a plastic liner or pouch 114 positioned within the cavity 104 and reservoir 106 is prefilled with a liquid substance 118. The plastic liner 114 may be formed as a pouch or puncturable plastic bag, with extensions 116 that extend into the reservoirs 106. In this embodiment, the teeth 102 may not have openings in their ends. Prior to use, the ends 110 of the teeth 102 are cut or snipped with a scissors or any type of shears or cutting instrument prior to use. Cutting adjacent the end 110 of a tooth 102 will simultaneously cut the portion of the extension 116 lining the reservoir 106 of that tooth 102, so that the liquid substance 18 will flow from the liner 114, and the applicator 100 may be used.

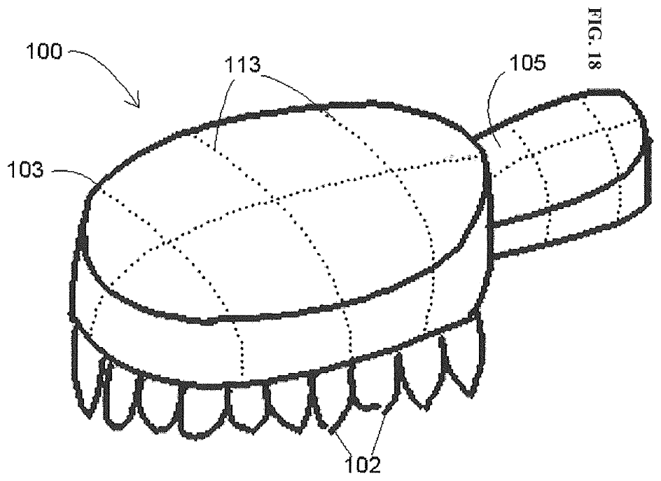

As shown in FIG. 18, to increase the breakability, the applicator 100 may further be formed with serrations, notches, cuts or fracture lines 113, along which the applicator 100 will break if sufficient force is supplied to the applicator 100, such as if an attempt is made to fashion the applicator 100 into a weapon. The facture lines 113 provide areas or portions where the material forming the applicator is weakened. Sufficient force may be, for example, the force applied in an attempt to shape the applicator 100 into a weapon, or use the applicator 100 as a weapon by attacking a person with the applicator 100. A child could also be given such an applicator, without fear of the child hurting them-selves, such that if the child pokes themselves too hard with the applicator, the applicator will break and/or fracture. Thus, a safety applicator is provided, having a novel and unique arrangement so that it can be given to a person where safety or fear of the person using a hard, non-breakable object, is a concern.

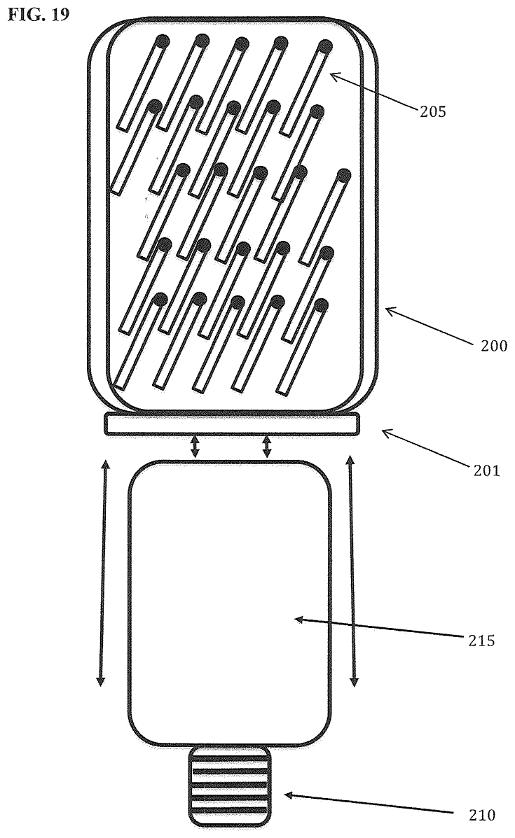

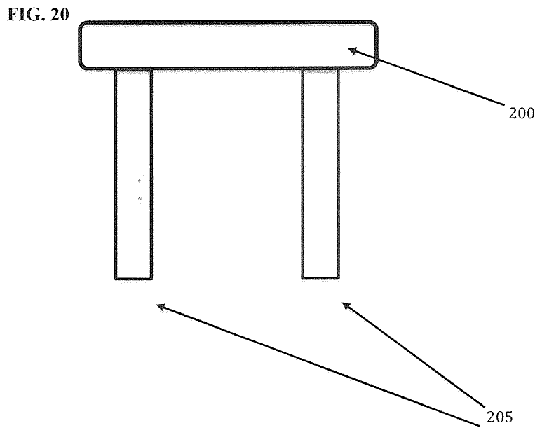

A still further embodiment is shown in FIG. 19 having a brush head 200 and an interlocking ergonomic engagement head 215 with grip 210. The applicator in the present invention alternatively may contain hollow bristles or teeth with liquid inserts 205 attached along the lower and/or upper surface of the brush head 200 having an interlocking slot 201 or other engagement means. The solid or flat surface portion 215 functions as an ergonomic handle grip which is inserted into the engagement slot 201 to secure the brush head for ease of application and holding. One or more individual bristles or teeth 205 attached to the brush head 200 shown in a cross-section on the lower and/or upper brush surface as represented in FIG. 20. Each of the bristles or teeth 205 has a hollow longitudinal portion extending to the liquid delivery portion with each containing a saturated felt tip or other means for dispensing liquid from within the hollow chamber. The felt tip allows for dispensing liquid to the skin surface during use. The engagement portion 215 can be released from the brush head 200 for separating the individual components for storage and reuse.

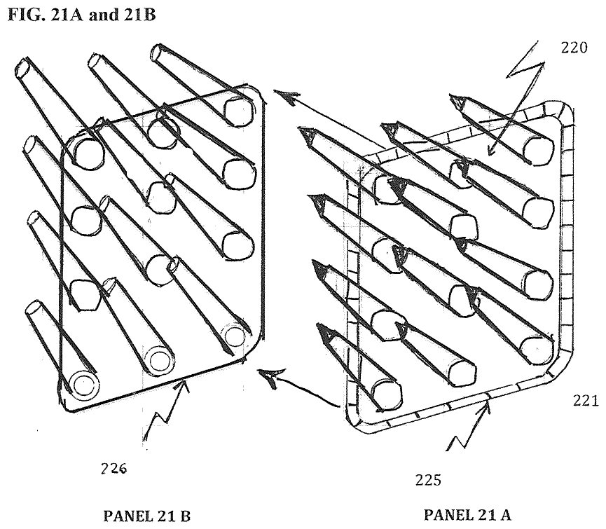

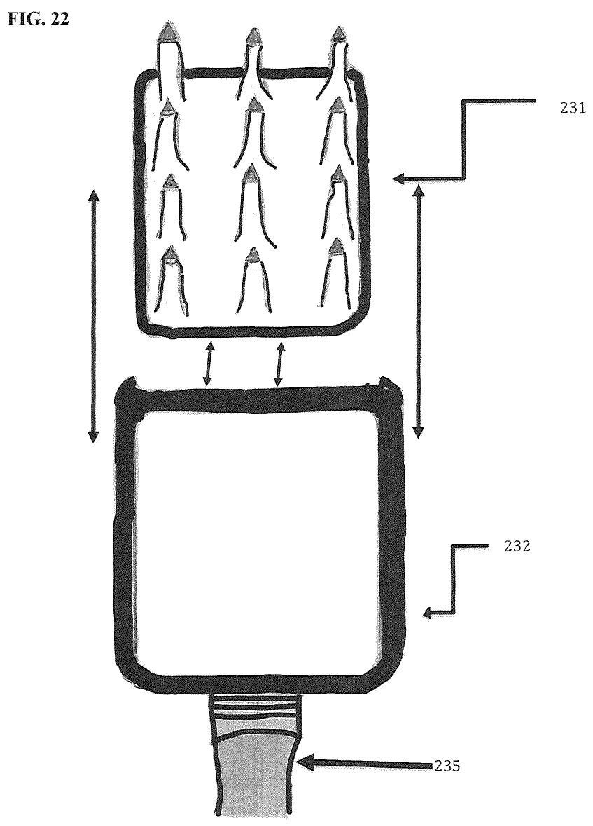

A further embodiment of the present invention utilizes a felt packet (220) with connected felt teeth insert, all as one piece and as shown in FIG. 21A and FIG. 21B. The Panel 21A shows the Felt packet incorporates the securing cap (225) to connect the felt packet to a solid attachment piece. Here, individual, liquid saturated teeth are inserted into the corresponding attachment as shown in (226) FIG. 21B. The attachment is a solid, rigid, disposable, and removable attachment as shown FIG. 22 provides a further perspective with respect to the orientation of the solid, rigid, disposable and removable attachment with the felt packet (231). A solid handle (235) allows receipt of the disposable, removable attachment with felt packet insert (232).

A still further embodiment of the present invention incorporates electronics and software into the brush head interfaced with a cell phone or similar computer device for analysis of the scalp or skin to detect if the skin is oily, dry or neutral. The brush provides high definition skin data to detect moisture, elasticity, oily, melanin, freckle and other skin or hair health issues. Hair follicle scalp detection from a digital microscope and hair follicle scalp detector allows image detection and analysis from the brush surface which sends data to an individual's cell phone or electronic device for further processing via an App. The brush head contains a high-performance sensor clip with a 24 bit DSP chip to provide 50×-500× magnification with built-in photo/video capabilities. An Auxiliary light source provides digital zooming options with an image distance from 0-40 mm from the scalp surface. Image resolution of 640-480 allows close examination of hair follicles. While not being limited, a still further embodiment exemplifying the practicality of the present devices, otherwise known as the Groomate, which incorporates the use of the applicator in the administration of a formula as a cosmetic composition having the effect of preventing and improving damage to hair and more specifically, but not limited to, aiding in dyeing or perm treatment using a composition that results in pure, recuperative, natural oils. When used with the Groomate, the liquid formula can be blended and evenly applied to the skin of a human or animal. The composition in liquid form contains, in part, ingredients vitamin E oil (approx. 10 ml), cotton seed oil (approx. 5 ml), aloe vera oil (approx. 10 ml), lanolin oil (approx. 10 ml), lavender oil (approx. 2.5 ml), vanilla bean oil (approx. 2.5 ml), clove oil (approx. 5 ml). The present invention further considers these proportions in a formulation designed uniquely to prevent damage to hair and to improve these conditions.

Hair can be susceptible to being easily damaged by the external environment. Consumers perceive hair as a fashion, the coloring of hair (dyeing) or using a procedure in a perm may contribute to this damage. In addition, hair damage may occur with exposure due to excessive use of a dryer or ultraviolet rays, all in a kind of lifestyle and other environmental factors that results in consumers damaging their hair.

Further with repetitive hair drying, characteristics such as shine, softness, and smoothness are deteriorated and hair styling becomes difficult.

The formulation disclosed herein, when used in conjunction with the device disclosed reduces these adverse affects and allows for improved consumer satisfaction.

It is appreciated that all of the various features of an applicator described herein may be combined to produce an applicator having a plurality of features such as described herein. Having thus described in detail several embodiments of the present invention, it is to be appreciated and will be apparent to those skilled in the art that many physical changes, only a few of which are exemplified in the detailed description of the invention, could be made without altering the inventive concepts and principles embodied therein. It is also to be appreciated that numerous embodiments incorporating only part of the preferred embodiment are possible which do not alter, with respect to those parts, the inventive concepts and principles embodied therein. The present embodiment and optional configurations are therefore to be considered in all respects as exemplary and/or illustrative and not restrictive, the scope of the invention being indicated by the appended claims rather than by the foregoing description, and all alternate embodiments and changes to this embodiment which come within the meaning and range of equivalency of said claims are therefore to be embraced therein.

1. A method for dispensing a liquid substance onto a subject's hair or skin comprising:
a. prefilling an applicator comprising:
i. a handle portion having an engagement portion;
ii. a felt tip dispenser having a hollow interior space for holding a liquid substance and a liquid substance held at least partially within the hollow interior space,
b. dispensing the liquid substance; and
c. engaging the dispenser removably engageable with the engagement portion of the handle portion,
wherein the liquid substance is released onto the subjects hair or skin.

2. The method of claim 1, wherein the liquid substance comprises a substance selected from the group consisting of pharmaceutical agents and supplements.

3. The method of claim 1, wherein the liquid substance comprises a composition containing vitamin E oil, cotton seed oil, aloe vera oil, lanolin oil, lavender oil, vanilla bean oil, clove ingredients and combinations thereof.

4. The method of claim 1 wherein the handle portion further comprises an actuator and a reservoir located within the interior space, the liquid substance held within the reservoir, the actuator in communication with the reservoir, the actuator adapted to cause the reservoir to selectively dispense the liquid substance when actuated.

5. The method of claim 1, wherein the dispenser comprises a removable cartridge.

6. The method of claim 1, wherein the dispenser comprises at least one removable ampule.

7. The method of claim 5, further comprising at least one tooth having a reservoir therein, the reservoir in communication with the hollow interior space via the at least one opening.

8. The method of claim 7, wherein the at least one tooth further comprises an open, application end for selectively dispensing the liquid substance.

9. The method of claim 8 further comprising a plug engageable with the application end, the plug configured to control passage of liquid from the reservoir of the at least one tooth.

10. The method of claim 6 further comprising a plug engageable with the at least one opening, the plug configured to control passage of liquid from the at least one ampule.

11. The method of claim 9 further comprising caps fittable over the at least one opening.

12. The method of claim 1 further having an optimum back-and-forth motion.

13. The method of claim 1 wherein the applicator has a breakable body comprising an internal cavity comprising a liquid substance and a handle.

14. The method of claim 13, wherein the applicator is formed from a foam material.

15. The method of claim 13, further comprising an open, application end for selectively dispensing the liquid substance and a cover formed to cover the application end.

16. The method of claim 13, wherein the applicator further comprises fracture lines providing areas along which the applicator will fracture upon the application of sufficient force.

17. The method of claim 1 further having electronic and image analysis software in the brush head to capture scalp and skin images wherein analysis detects moisture, elasticity, oily, melanin skin conditions.

18. The method of claim 16 having image analysis software that provide analysis for moisture, elasticity oily, melanin skin conditions to the subjects app.

19. The method of claim 1 where in the subject is selected from the group consisting of a person, a pet, a fur bearing animal, a feather bearing animal, or a combination therein.

20. A device for dispensing a liquid substance onto a subject's hair or skin comprising:
a. an applicator capable of prefilling comprising:
i. a handle portion having an engagement portion;
ii. a felt tip dispenser having a hollow interior space for holding a liquid substance and a liquid substance held at least partially within the hollow interior space,
b. a liquid substance dispenser; and
c. a means for engaging the dispenser with the engagement portion of the handle portion,
wherein a liquid substance is released onto a subject's hair or skin.
